(19) United States
(12) Patent Application Publication
(10) Pub. No.: US 2004/0219552 A1
(43) Pub. Date: Nov. 4, 2004

(54) Novel molecular target for neurotoxicity

(76) Inventors: Ali Ait Ikhlef, Gomerz Le Cratel (FR); Annelies Resink, Paris (FR); Fabien Schweighoffer, Vincennes (FR)

Correspondence Address: NIXON & VANDERHYE, PC, 1100 N GLEBE ROAD, 8TH FLOOR, ARLINGTON, VA, 22201-4714, US

(21) Appl. No.: 10/486,639

(22) PCT Filed: Aug. 13, 2001
(86) PCT No.: PCT/FR02/02861

Related U.S. Application Data
(63) Continuation-in-part of application No. 09/983,754, filed on Oct. 25, 2001.

(30) Foreign Application Priority Data
Aug. 14, 2001 (FR) 01 10819

(51) Int. Cl. C12Q 1/68; G01N 33/53; G01N 33/567

(52) U.S. Cl. 435/6; 435/7.2

(57) ABSTRACT

The present invention relates to the fields of biology, genetics and medicine. In particular it concerns new methods for the detection, characterisation and/or treatment (or management) of neurodegenerative diseases, particularly amyotrophic lateral sclerosis. The invention equally concerns methods for identifying or screening compounds active in these diseases. The invention further concerns the compounds, genes, cells, plasmids or compositions useful for implementing the hereinabove methods. In particular, the invention describes the role of PDE4B in these diseases and its use as a therapeutic, diagnostic or experimental target.

The present invention relates to the fields of biology, genetics and medicine. In particular it concerns new methods for the detection, characterisation and/or treatment (or management) of neurodegenerative diseases, particularly amyotrophic lateral sclerosis. The invention equally concerns methods for identifying or screening compounds active in these diseases. The invention further concerns the compounds, genes, cells, plasmids or compositions useful for implementing the hereinabove methods. The invention derives notably from the identification of the role of phosphodiesterase 4B in these diseases and describes its use as target or therapeutic, diagnostic or experimental marker in these disorders.

Many neurodegenerative diseases have been described as having a component or a stage linked to the phenomenon of excitotoxicity. Such is the case for Alzheimer's disease, Parkinson's disease, multiple sclerosis and Huntington's chorea.

Amyotrophic lateral sclerosis (or ALS) is a neurodegenerative disease accompanied by different types of inclusions such as Lewis bodies and characterised by apoptosis of spinal and cortical motor neurons whose death is sometimes associated with frontal dementia. Sporadic forms for which no mutation has been described exist alongside familial forms (FALS) associated with mutations in the SOD1 gene encoding superoxide dismutase. The majority of cases is sporadic, familial forms (FALS) being very rare. It is likely that a long, asymptomatic period precedes the onset of clinical symptoms, which are variable and difficult to classify. Future advances in therapy will make it possible to replace symptomatic treatments with strategies based on the molecular causes of the disease. At the cellular level, these symptoms are related to death of cortical motor neurons and spinal motor neurons. This neuronal death has been linked to different phenomena which underlie a number of neurodegenerative diseases. Such is the case of excitotoxicity linked to glutamate, oxidative stress, auto-immunity directed against neuronal markers (calcium channels in the case of ALS) as well as cytoskeletal abnormalities. Although such phenomena are known, the cause or causes of these diseases, including ALS, remain obscure. Even though FALS is related to mutations in the SOD1 gene coding for superoxide dismutase, the mechanisms by which neurons become committed towards cellular death, of which at least one component is apoptosis, are unknown.

Elucidating the molecular events involved in the different phenomena implicated in cell death will allow the development of new therapeutic strategies. The study of these events is difficult to carry out using human biopsy specimens. Such biopsies obviously come from post-mortem samples whose quality is difficult to control and which reflect only the pathological states present at the late stages of the disease.

Animal models give access to biological samples that allow the different steps of disease development to be analysed and compared with healthy controls. In this respect, transgenic mice expressing the human SOD1 gene bearing one of the mutations prevalent in FALS (mutation G93A) are available from Jackson Laboratory, on condition that a user's licence is obtained from Northwestern University. This model reproduces in 120 days the fatal outcome of the disease with symptoms similar to those in the human disease. The onset of ALS symptoms related to mutation G93A in the SOD1 gene does not result from a reduction in superoxide dismutase activity but rather a gain in function which increases the capacity of the enzyme to generate free radicals. Despite this knowledge, the molecular events governing the different stages of ALS are poorly understood. The complexity of these molecular events reflects the progression of the disease: in the transgenic model studied, no neuronal deregulation or clinical manifestations are observed at 30 days. Sixty days is a stage shortly before symptom onset, but which is already characterised in brain by changes in cellular physiology such as alteration of mitochondrial metabolism, stress and neuronal death associated with an excitotoxicity phenomenon. At 90 days, 50% of cortical and spinal motor neurons are dead and an active process of neuronal apoptosis begins in parallel to an activation of astrocytes. The phenomenon of excitotoxicity is no longer observed at this stage. Neuronal death is associated with activation of caspases which do not appear to be involved in the early stages of the disease.

Elucidating the different molecular events specific of the different stages of the disease should allow identification of new therapeutic targets as well as new diagnostic markers. One of the most effective approaches to carry out this identification consists in identifying the genes and proteins whose expression characterises a pathophysiological state.

The present invention now describes the identification of genetic events involved in the phenomena of excitotoxicity and neuronal death. The present invention thus provides new therapeutic and diagnostic approaches to the diseases associated with these phenomena, as well as new targets for identifying active compounds.

More particularly, a qualitative differential analysis has been carried out on RNA extracted from brain and spinal cord samples without preliminary isolation of neurons in order to take into account a maximum of alternative splicing events related to disease development. This analysis was carried out by qualitative differential screening according to the DATAS method (described in application No. WO99/46403), which has unequalled advantages.

The present patent application is derived in particular from the applicant's construction of a repertoire of alternative splicings in the brains of 60-day-old animals in the ALS model. This repertoire, which contains more than 200 separate sequences, comprises key players in the excitotoxicity phenomenon, such as potassium channels and the NMDA receptor. Sequences derived from RNAs coding for proteins involved in the response to stress, including heat shock proteins, are also part of this repertoire, underscoring the role of this latter response in the early stages of ALS. Altered energy metabolism clearly appears to affect cortical motor neurons of animals that develop the disease. For instance, intron 6 of mitochondrial creatine kinase is isolated specifically from messenger RNAs expressed in pathological conditions in 60-day-old animals. Interruption of the coding sequence by retention of this intron results in a messenger RNA that encodes an inactive form of the enzyme. This observation agrees with biochemical findings showing a reduction of mitochondrial creatine kinase activity correlated with a reduction in the amount of this enzyme in neurons from animals in the same transgenic model.

The specificity of the sequences making up this repertoire is confirmed by the fact that the same qualitative differential analysis of gene expression performed in 90-day-old animals leads to a different repertoire in which, in particular, the different markers of excitotoxicity are absent. Analysis of splicing modifications confirms that the molecular events differ according to the stage of the disease.

In a particularly interesting and unexpected manner, the use of DATAS on RNA from 60-day-old transgenic and control animals has led to the isolation of a cDNA fragment derived from the mRNA of phosphodiesterase 4B. Such fragment corresponds to an exon fragment specifically present in control animals and therefore specifically deleted in SOD1G93A transgenic animals at the 60 day stage. Such fragment spans nucleotides 377 to 486 numbered from the mouse PDE48 stop codon (SEQ ID NO:1) (sequence also accessible in GenBank, No. AF208023). This sequence comprises 2912 bases, the deleted fragment corresponding to bases 2760 to 2869. This is a noncoding region and is differentially expressed in control animals and transgenic animals due to the alternative use of a noncoding 3′ exon or due to the use of two alternative polyadenylation sites. This differential expression has been demonstrated by the RT-PCR experiments presented in FIGS. 1A and 1B.

The present application therefore demonstrates the involvement of phosphodiesterase 4B in the development of excitotoxicity processes and neuronal death. The results obtained reveal a higher level of expression of PDE4B in pathological nerve tissue, in relation to a structural modification of the corresponding RNA, more particularly the deletion of a region in the 3′ noncoding part. This result is altogether compatible with the presence of mRNA destabilisation sequences in the sequence identified by DATAS. Their deletion in PDE4B mRNA, through splicing or through the use of alternative polyadenylation sequences, can result in stabilisation, therefore in an increased expression of the coding portion of this RNA. This event occurs specifically in the brain of pathological subjects and not in control subjects.

The present invention therefore describes an original molecular event leading to increased expression of PDE4B mRNA in the brain of pathological subjects and which is correlated over time with the phenomenon of excitotoxicity and/or neuronal death. The invention further shows, for the first time, that increased expression of PDE4B is associated with the early stages of ALS. PDE4B is therefore a new and important therapeutic target in the development of treatments for these diseases, of particular use in the early stages of their development, and addressing the true molecular bases of the disease and not the accompanying symptoms or inflammatory components. The invention also provides for new methods of diagnosis, screening, detection, determination of a predisposition or monitoring the progression or the efficacy of treatment of these diseases.

Detection, Diagnosis and Screening

One object of the invention is therefore to provide a method for detecting an excitotoxicity situation or neuronal stress in a subject, comprising measuring in vitro the expression of phosphodiesterase 4, particularly phosphodiesterase 4B, in a sample from the subject. The method advantageously comprises measuring the differential expression of the 3′ noncoding region of the PDE4B gene and the rest of the gene, particularly the coding portion.

A further object of the invention is therefore to provide a method for detecting an excitotoxicity situation or neuronal stress in a subject, comprising detecting the presence of a mutant RNA of phosphodiesterase 4, particularly phosphodiesterase 4B, in a sample from the subject, in particular a form deleted of all or part of the 3′ noncoding region.

Another object of the invention is the use of a nucleic acid comprising all or part of a sequence derived from the PDE4B gene or messenger RNA for implementing a method for diagnosis or detection of a situation of neuronal stress and more specifically an excitoxicity situation.

The invention is generally based on the use of a nucleic acid complementary to all or part of the PDE4B gene or messenger, for detecting pathological events related to excitotoxicity, stress, neuronal death, etc. More generally, the invention provides a method for the diagnosis, screening, characterisation or monitoring of a degenerative disease, comprising demonstrating an alteration in the PDE4 gene or in the corresponding RNA, typically PDE4B.

The expression of PDE4, or the differential expression, or the presence of an altered form, may be determined by conventional methods of molecular biology, such as for example sequencing, hybridisation, amplification, RT-PCR, gel migration, and the like. The invention has applications in the diagnosis or detection of different pathologies involving excitotoxicity phenomena, such as Alzheimer's disease, Parkinson's disease, multiple sclerosis, ALS, Huntington's chorea or cerebral ischemia. It may be used for early detection, to demonstrate a predisposition, to guide the choice and adaptation of a treatment, to monitor disease progression, etc. It is especially suited to detecting multiple sclerosis or ALS at an early stage.

To implement the genetic methods of diagnosis or detection according to the invention, one more particularly uses nucleic acids capable of demonstrating a deleted form of PDE4B mRNA, particularly a form deleted of all or part of the 3′ noncoding region. A specific example is the use of a nucleic acid complementary to all or part of the region located between residues 2760 to 2869 of sequence SEQ ID No.: 1, or corresponding residues of the sequence of the human PDE4B gene or mRNA. The cDNA sequence encoding human PDE4B and the corresponding protein are shown in sequences SEQ ID No.: 3 and 4 (also see Genbank, No. NM_{—}002600). The 3′ noncoding region of the human PDE4B gene or RNA corresponds to residues 2461 to 4068 of SEQ ID No.: 3.

In an advantageous manner, the nucleic acid used (as probe) comprises all or part of the sequence coding for the 3′ noncoding region of the PDE4B gene or RNA located between nucleotides 2384 and 2869 of the sequence SEQ ID NO.: 1 or between nucleotides 2461 and 4068 of the sequence SEQ ID NO: 3 or a sequence complementary thereto.

According to specific embodiments, the invention makes use of a nucleic acid complementary to a region located within one of the following sequences:

residues 2384 to 2869 of SEQ ID NO 1

residues 2500 to 2869 of SEQ ID NO 1

residues 2760 to 2869 of SEQ ID NO 1

residues 2780 to 2850 of SEQ ID NO 1

residues 2790 to 2810 of SEQ ID NO 1

residues 2600 to 4040 of SEQ ID NO 3

residues 3000 to 4040 of SEQ ID NO 3

residues 3500 to 4040 of SEQ ID NO 3

residues 3900 to 4040 of SEQ ID NO 3.

In another specific embodiment, one uses a nucleic acid complementary to the sequence of the PDE4 RNA region resulting from deletion of all or part of the 3′ noncoding part. Deletion of a domain in fact creates new junctions in the sequence, which are specific of the deleted form and may be used to demonstrate the presence of such a form in a sample.

Preferably, the degree of complementarity between the probe and the target sequence is perfect so as to ensure better specificity of hybridisation. However, it is understood that some mispairing may be tolerated. The nucleic acid used for implementation of the hereinabove methods may be a DNA or an RNA, preferably a synthetic DNA. It preferably comprises 10 to 500 bases, typically 10 to 100 bases. It is understood that a longer nucleic acid may be used, if desired, although this is not preferred. The nucleic acid is advantageously a single stranded DNA, from 10 to 500 bases, complementary at least to a region of the 3′ noncoding sequence of PDE4B. The nucleic acid may be labelled, for instance by radioactivity, enzymatic, luminescent, fluorescent, chemical means, etc.

Another approach for detecting the presence of an alteration in the PDE4 gene makes use of a primer or a nucleic primer pair allowing selective amplification of a portion of PDE4 RNA, preferably comprising a portion of the 3′ noncoding region. One typically uses a primer allowing selective amplification of the altered form of PDE4 RNA, particularly a primer specific of the junction created by deletion of part of the RNA 3′ region.

In this regard, one object of the invention is based on a primer complementary to a portion of the PDE4B 3′ noncoding region, and allowing amplification of a part of this region. The primer advantageously comprises 8 to 20 bases. It is preferably composed of a fragment of 8 to 20 consecutive residues of the sequence located between nucleotides 2384 and 2869 of sequence SEQ ID NO: 1 or between nucleotides 2461 and 4068 of the sequence SEQ ID NO: 3 or a sequence complementary thereto. A further object of the invention is a primer pair allowing specific amplification of at least part of the PDE4 3′ noncoding region, said pair comprising at least one primer such as defined hereinabove.

To implement the methods according to the invention, a biological sample from a subject, containing a nucleic acid, is placed in contact in vitro with a nucleic acid (probe, primer, etc.) such as defined hereinabove, and the formation of a hybrid or an amplified product is detected. The biological sample may be a sample of blood, fluid, cell, tissue, etc. The nucleic acid may be immobilised on a support of the type glass, silica, nylon, etc.

The process of detection, screening or diagnosis may be implemented by using different types of samples from a subject, such as for instance tissue biopsies, particularly nerve tissue. In an especially surprising and advantageous manner, the present invention further shows that deregulation of PDE4 expression, correlated with the excitotoxicity phenomenon, may be directly demonstrated in muscle tissue. This is especially remarkable in the case of neurodegenerative diseases such as ALS.

During the development of ALS, degenerative phenomena occur not only in brain but also in spinal cord and consequently in muscle through defective innervation. FIG. 2 depicts the modifications of PDE4B mRNA expression in muscle from control and transgenic mice, detected by using the same PCR primers as in the experiment on RNA from the brains of these same animals. In an analogous, but less pronounced manner, a reduction in the expression of the 3′ noncoding region of PDE4B, and not in the remainder of this mRNA (particularly the coding portion), is observed specifically in muscle of animals at the end of the presymptomatic stage, i.e. aged 90 days.

One difficulty encountered in the study and treatment of ALS is that of establishing an early diagnosis. The observation that PDE4B mRNA is deregulated in ALS muscle makes it possible to establish an early diagnosis from muscle biopsies of patients. Such diagnosis is based on the detection of differential expression between the 3′ noncoding region and the rest of the sequence, particulary the coding portion, of PDE4B.

A specific method for detecting a situation of neuronal stress, notably excitotoxicity, in particular linked to a neurodegenerative disease in a subject, comprises measuring PDE4B gene expression, or the presence of deleted forms of the PDE4B messenger, in a sample of muscle cells from said subject.

To measure differential expression, one uses for example a probe corresponding to (that is to say, specific of) a part of the 3′ noncoding region and a probe corresponding to a part of the coding region of PDE4B. The signal detected with each of these probes allows an evaluation of differential expression. Another approach makes use of two primer pairs allowing amplification of a portion of the 3′ noncoding region on the one hand and a portion of the coding region on the other hand.

An additional object is a kit for analysing PDE4 expression, particularly the differential expression between the 3′ noncoding region and the coding region, the kit comprising a nucleotide probe specific of a part of the sequence of the 3′ noncoding region and a nucleotide probe specific of a part of the sequence of the coding region.

A further object is a kit for analysing PDE4 expression, particularly the differential expression between the 3′ noncoding region and the coding region, the kit comprising a pair of nucleotide primers allowing specific amplification of at least part of the 3′ noncoding region of PDE4 and a pair of nucleotide primers allowing specific amplification of at least part of the coding region of PDE4.

Therapy

Phosphodiesterases hydrolyse cyclic nucleic acids such as cAMP and cGMP, regulating different signalling cascades. PDE4B hydrolyses cAMP, thereby regulating the concentrations of this second messenger inside the cell. The role of cAMP in the balance between cell viability and apoptosis has been well described in the literature. In particular, the cAMP cascade plays an integral role in cell survival cascades involving kinases like Akt and Pl3K as well as in regulating the activity of transcription factor CREB. It is noteworthy that this transcription factor is involved in neuron survival and neurite growth. Nonetheless, the use of PDE and, advantageously, PDE4 inhibitors has never been envisioned to improve neuron viability and more particularly to protect them against excitotoxicity. It has been suggested that PDE4 inhibitors, developed to inhibit inflammatory phenomena, may potentially be useful in neurodegenerative diseases such as Alzheimer's disease. This suggestion is based on the goal of reducing the inflammation observed in brain during neurodegenerative processes and not at all on a rationale aiming to directly inhibit neuronal death.

The present invention demonstrates the existence of splicing events and alternative polyadenylation sites affecting the PDE4B gene, associated with the development of neuronal excitotoxicity, and provides the molecular basis that justifies the use of PDE4 inhibitors for the treatment of ALS and more generally for improvement of neuron viability during excitotoxicity phenomena, in particular starting from the early stages of these diseases.

Another object of the invention is therefore based on the use of a compound capable of inhibiting or reducing the expression or activity of PDE4B, in order to prepare a composition designed to treat neurodegenerative diseases, notably in early stages, more preferably to reduce the early neuronal excitotoxicity associated with neurodegenerative diseases such as ALS, Alzheimer's disease or Parkinson's disease.

A particular object consists in the use of a PDE4 inhibitor for preparing a composition designed to treat ALS, particularly to reduce excitotoxicity in subjects with ALS or to increase neuron survival in subjects with ALS.

Another object of the invention is the use of a compound capable of inhibiting (preferably in a selective manner) the expression or activity of PDE4B of sequence SEQ ID NO: 2 or 4 in order to prepare a composition designed to reduce neuronal excitotoxicity.

A further object of the invention is a method for treating a disease associated with neuronal stress, particularly excitotoxicity, comprising administering to a subject a compound that inhibits PDE4B activity or expression, preferably a compound that selectively inhibits PDE4.

Another object of the invention is based on a method for treating ALS, particularly a method for increasing neuron survival in subjects with ALS, comprising administering to a subject a PDE4 inhibitor, preferably a compound that selectively inhibits PDE4.

Within the context of the invention, the term “treatment” refers to preventive, curative, palliative treatment, as well as management of patients (alleviating suffering, improving life expectancy, slowing disease progression), etc. The treatment may furthermore be conducted in combination with other agents or treatments, especially addressing late events in the disease, such as caspase inhibitors or other active compounds.

The compound used may be any compound that can inhibit the expression of PDE4, particularly PDE4B, that is to say, in particular any compound inhibiting gene transcription, RNA maturation, mRNA translation, posttranslational protein modification, etc. It may be a compound inhibiting RNA modification, particularly the deletion of part of the 3′ noncoding region.

In a specific embodiment, the compound is an antisense nucleic acid, capable of inhibiting transcription of the PDE4B gene or translation of the corresponding mRNA. The antisense nucleic acid may comprise all or part of the sequence of the PDE4B gene, a fragment thereof, the PDE4B messenger, or a sequence complementary thereto. The antisense nucleic acid may notably comprise a region complementary to the sequence located between residues 218 to 2383 of SEQ ID NO:1 or 766 to 2460 of SEQ ID NO: 3, and inhibit (or reduce) its translation into protein. The antisense nucleic acid may be a DNA, an RNA, a ribozyme, etc. It may be single-stranded or double-stranded. It may also be an RNA encoded by an antisense gene. Where it is an antisense oligonucleotide, it typically contains fewer than 100 bases, for example on the order of 10 to 50 bases. Such oligonucleotide may be modified to improve its stability, its resistance to nucleases, its penetration into the cell, etc.

According to a further embodiment, the compound is a peptide, for example comprising a region of the PDE4 protein (particularly PDE4B) and able to antagonise its activity.

According to another embodiment, the compound is a chemical compound of natural or synthetic origin, particularly an organic or inorganic molecule, of plant, bacterial, viral, animal, eukaryotic, synthetic or semi-synthetic origin, capable of modulating the expression or activity of PDE4B.

In a preferred variant, the compound is a synthetic compound that inhibits PDE4. Different types of inhibitors may be used. Preferably they are compounds from the pyrazolopyridine family, among which a specific example is etazolate, or compounds from the family of xanthine (or 2,6-dioxopurine) derivatives, including in particular pentoxifylline.

Compounds from the pyrazolopyridine family are chosen in particular from among the following compounds:

Etazolate which has the following formula:

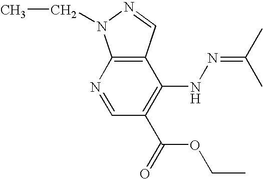

4-butylamino-1-ethyl-6-methyl-1H-pyrazolo[3,4-b]pyridine-5-carboxylic acid ethyl ester (tracazolate),

4-butylamino-1-ethyl-1H-pyrazolo[3,4-b]pyridine-5-carboxylic acid ethyl ester,

1-(4-amino-pyrazolo[3,4-b]pyridin-1-yl)-β-D-1-deoxy-ribofuranose,

1-ethyl-4-(N′-isopropylidene-hydrazino)-1H-pyrazolo[3,4-b]pyridine-5-carboxylic acid ethyl ester (SQ 20009),

4-amino-6-methyl-1-n-pentyl-1H-pyrazolo[3,4-b]pyridine,

4-amino-1-ethyl-6-methyl-1H-pyrazolo[3,4-b]pyridine-5-carboxylic acid ethyl ester (desbutyl tracacolate),

4-amino-1-pentyl-1H-pyrazolo[3,4-b]pyridine-5-carboxamide,

1-ethyl-6-methyl-4-methylamino-1H-pyrazolo[3,4-b]pyridine-5-carboxylic acid ethyl ester,

4-amino-6-methyl-1-propyl-1H-pyrazolo[3,4-b]pyridine-5-carboxylic acid ethyl ester,

1-ethyl-4-ethylamino-6-methyl-1H-pyrazolo[3,4-b]pyridine-5-carboxylic acid ethyl ester,

4-amino-1-butyl-6-methyl-1H-pyrazolo[3,4-b]pyridine-5-carboxylic acid ethyl ester,

5-(4-amino-pyrazolo[3,4-b]pyridin-1-yl)-2-hydroxymethyl-tetrahydro-furan-3-ol,

1-allyl-4-amino-6-methyl-1H-pyrazolo[3,4-b]pyridine-5-carboxylic acid allyl ester,

4-amino-6-methyl-1-pentyl-1H-pyrazolo[3,4-b]pyridine-5-carboxylic acid,

4-amino-1-ethyl-3,6-dimethyl-1H-pyrazolo[3,4-b]pyridine-5-carboxylic acid ethyl ester,

4-dimethylamino-1-ethyl-6-methyl-1H-pyrazolo[3,4-b]pyridine-5-carboxylic acid ethyl ester,

1-ethyl-6-methyl-4-propylamino-1H-pyrazolo[3,4-b]pyridine-5-carboxylic acid ethyl ester,

4-amino-1-pentyl-1H-pyrazolo[3,4-b]pyridine-5-carboxylic acid ethyl ester,

4-amino-6-methyl-1-pent-4-ynyl-1H-pyrazolo[3,4-b]pyridine-5-carboxylic acid ethyl ester,

4-amino-1-but-3-enyl-1H-pyrazolo[3,4-b]pyridine-5-allylamide,

4-amino-1-pentyl-1H-pyrazolo[3,4-b]pyridine-5-isopropylamide,

4-amino-1-pentyl-N-n-propyl-1H-pyrazolo-[3,4-b]pyridine-5-carboxamide,

4-amino-1-butyl-6-methyl-1H-pyrazolo[3,4-b]pyridine-5-carboxylic acid allyl ester,

4-amino-6-methyl-1-pent-3-ynyl-1H-pyrazolo[3,4-b]pyridine-5-carboxylic acid ethyl ester,

4-amino-1-pentyl-1H-pyrazolo[3,4-b]pyridine-5-prop-2-ynylamide,

4-amino-1-(3-methyl-butyl)-1H-pyrazolo[3,4-b]pyridine-5-carboxylic acid allyl ester,

4-amino-1-pentyl-1H-pyrazolo<3,4-b>pyridine-5-N-(2-propenyl)carboxamide,

4-amino-1-pentyl-1H-pyrazolo[3,4-b]pyridine-5-carboxylic acid allyl ester,

4-amino-1-pentyl-1H-pyrazolo[3,4-b]pyridine-5-butylamide,

4-amino-1-but-3-ynyl-6-methyl-1H-pyrazolo[3,4-b]pyridine-5-carboxylic acid allyl ester,

4-amino-1-but-3-enyl-6-methyl-1H-pyrazolo[3,4-b]pyridine-5-carboxylic acid allyl ester,

4-amino-6-methyl-1-pentyl-1H-pyrazolo[3,4-b]pyridine-5-allylamide,

4-amino-6-methyl-1-pentyl-1H-pyrazolo[3,4-b]pyridine-5-carboxylic acid allyl ester,

4-amino-6-methyl-1-(3-methyl-butyl)-1H-pyrazolo[3,4-b]pyridine-5-carboxylic acid allyl ester,

4-amino-6-methyl-1-pentyl-1H-pyrazolo[3,4-b]pyridine-5-carboxylic acid isobutyl ester,

4-amino-6-methyl-1-pentyl-1H-pyrazolo[3,4-b]pyridine-5-butylamide,

4-amino-6-methyl-1-(3-methyl-but-2-enyl)-1H-pyrazolo[3,4-b]pyridine-5-carboxylic acid allyl ester,

4-amino-1-pentyl-1H-pyrazolo[3,4-b]pyridine-5-cyclopropylamide,

ethyl 4-amino-1-pentyl-1H-pyrazolo[3,4-b]pyridine-5-hydroxamate,

4-amino-6-methyl-1-pentyl-1H-pyrazolo[3,4-b]pyridine-5-carboxylic acid prop-2-ynyl ester,

4-amino-6-methyl-1-pent-4-ynyl-1H-pyrazolo[3,4-b]pyridine-5-carboxylic acid allyl ester,

4-amino-6-methyl-1-pent-4-enyl-1H-pyrazolo[3,4-b]pyridine-5-carboxylic acid allyl ester,

4-amino-1-pent-3-ynyl-1H-pyrazolo[3,4-b]pyridine-5-propylamide,

4-amino-1-pentyl-1H-pyrazolo[3,4-b]pyridine-5-cyclopropylmethyl-amide,

4-amino-6-methyl-1-pentyl-1H-pyrazolo[3,4-b]pyridine-5-carboxylic acid 2-methyl-allyl ester,

4-amino-1-pent-3-ynyl-1H-pyrazolo[3,4-b]pyridine-5-allylamide (ICI 190,622),

4-amino-1-pent-4-ynyl-N-2-propenyl-1H-pyrazolo[3,4-b]pyridine-5-carboxamide,

4-amino-1-pent-3-ynyl-1H-pyrazolo[3,4-b]pyridine-5-prop-2-ynylamide,

4-amino-1-pentyl-1H-pyrazolo[3,4-b]pyridine-5-but-2-ynylamide,

4-amino-6-methyl-1-pent-3-ynyl-1H-pyrazolo[3,4-b]pyridine-5-carboxylic acid allyl ester,

4-amino-1-(2-cyclopropyl-ethyl)-6-methyl-1H-pyrazolo[3,4-b]pyridine-5-carboxylic acid allyl ester,

4-amino-1-hex-5-ynyl-6-methyl-1H-pyrazolo[3,4-b]pyridine-5-carboxylic acid allyl ester,

4-amino-1-pent-3-ynyl-1H-pyrazolo[3,4-b]pyridine-5-cyclopropylmethyl-amide,

4-amino-6-methyl-1-pentyl-1H-pyrazolo[3,4-b]pyridine-5-carboxylic acid but-3-enyl ester,

4-amino-6-methyl-1-pentyl-1H-pyrazolo[3,4-b]pyridine-5-carboxylic acid cyclopropylmethyl ester,

4-butylamino-1-pentyl-1H-pyrazolo[3,4-b]pyridine-5-allylamide,

4-amino-6-methyl-1-pentyl-1H-pyrazolo[3,4-b]pyridine-5-carboxylic acid 2-cyclopropyl-ethyl ester,

4-amino-6-methyl-1-pent-3-ynyl-1H-pyrazolo[3,4-b]pyridine-5-carboxylic acid cyclopropylmethyl ester,

4-amino-6-methyl-1-pent-4-ynyl-1H-pyrazolo[3,4-b]pyridine-5-carboxylic acid cyclopropylmethyl ester,

4-amino-1-benzyl-6-methyl-1H-pyrazolo[3,4-b]pyridine-5-carboxylic acid ethyl ester,

4-amino-1-pentyl-1H-pyrazolo[3,4-b]pyridine-5-benzylamide,

4-amino-1-pentyl-1H-pyrazolo[3,4-b]pyridine-5-phenylamide,

4-amino-6-methyl-1-pentyl-1H-pyrazolo[3,4-b]pyridine-5-carboxylic acid benzyl ester,

4-azido-1-β-D-ribofuranosylpyrazolo[3,4-b]pyridine,

1-pent-3-ynyl-N-2-propenyl-4-propionamido-1H-pyrazolo[3,4-b]pyridine-5-carboxamide,

2-(4-amino-pyrazolo[3,4-b]pyridin-1-yl)-5-hydroxymethyl-tetrahydro-furan-3,4-diol,

2-(6-methyl-1H-pyrazolo[3,4-b]pyridin-4-ylamino)-ethanol,

3-(6-methyl-1H-pyrazolo[3,4-b]pyridin-4-ylamino)-propan-1-ol,

3-(6-methyl-1H-pyrazolo[3,4-b]pyridin-4-ylamino)-acetic acid propyl ester,

2-(6-methyl-1H-pyrazolo[3,4-b]pyridin-4-ylamino)-propionic acid ethyl ester,

2-(6-methyl-1H-pyrazolo[3,4-b]pyridin-4-ylamino)-pentanoic acid ethyl ester,

2-(6-methyl-1H-pyrazolo[3,4-b]pyridin-4-ylamino)-benzoic acid ethyl ester,

3-(6-methyl-1H-pyrazolo[3,4-b]pyridin-4-ylamino)-pentanoic acid propyl ester,

N-benzylidene-N′-(3-methyl-1-phenyl-1H-pyrazolo[3,4-b]pyridin-4-yl)-hydrazine,

N-furan-2-ylmethylene-N′-(3-methyl-1-phenyl-1H-pyrazolo[3,4-b]pyridin-4-yl)-hydrazine,

N-(4-fluoro-benzylidene)-N′-(3-methyl-1-phenyl-1H-pyrazolo[3,4-b]pyridin-4-yl)-hydrazine,

N-(3-furan-2-yl-allylidene)-N′-(3-methyl-1-phenyl-1H-pyrazolo[3,4-b]pyridin-4-yl)-hydrazine,

N-(4-methoxy-benzylidene)-N′-(3-methyl-1-phenyl-1H-pyrazolo[3,4-b]pyridin-4-yl)-hydrazine,

4-[(3-methyl-1-phenyl-1H-pyrazolo[3,4-b]pyridin-4-yl)-hydrazonomethyl]-benzonitrile,

N-benzo[1,3]dioxol-5-ylmethylene-N′-(3-methyl-1-phenyl-1H-pyrazolo[3,4-b]pyridin-4-yl)-hydrazine,

N-(3-methyl-1-phenyl-1H-pyrazolo[3,4-b]pyridin-4-yl)-N′-(4-nitro-benzylidene)-hydrazine,

N-(3-methyl-1-phenyl-1H-pyrazolo[3,4-b]pyridin-4-yl)-N′-(2-nitro-benzylidene)-hydrazine,

N-(3-methyl-1-phenyl-1H-pyrazolo[3,4-b]pyridin-4-yl)-N′-(4-trifluoromethyl-benzylidene)-hydrazine,

N-(3-methyl-1-phenyl-1H-pyrazolo[3,4-b]pyridin-4-yl)-N′-(5-nitro-furan-2-ylmethylene)-hydrazine,

N-(3-methyl-1-phenyl-1H-pyrazolo[3,4-b]pyridin-4-yl)-N′-(2-trifluoromethyl-benzylidene)-hydrazine,

N-(3-methyl-1-phenyl-1H-pyrazolo[3,4-b]pyridin-4-yl)-N′-(6-nitro-benzo[1,3]dioxol-5-ylmethylene)-hydrazine,

4-(3-chloro-4-methoxy-benzylamino)-1-ethyl-1H-pyrazolo[3,4-b]pyridine-5-carboxylic acid,

4-(3-chloro-4-methoxy-benzylamino)-1-ethyl-1H-pyrazolo[3,4-b]pyridine-5-(pyridin-4-ylmethyl)-amide,

4-(3-chloro-4-methoxy-benzylamino)-1-ethyl-1H-pyrazolo[3,4-b]pyridine-5-(tetrahydro-furan-2-ylmethyl)-amide,

4-(3-chloro-4-methoxy-benzylamino)-1-ethyl-1H-pyrazolo[3,4-b]pyridine-5-(5-hydroxy-pentyl)-amide,

4-(3-chloro-4-methoxy-benzylamino)-1-ethyl-1H-pyrazolo[3,4-b]pyridine-5-[3-(2-oxo-pyrrolidin-1-yl)-propyl]-amide,

4-tert-butylamino-1-(2-chloro-2-phenyl-ethyl)-1H-pyrazolo[3,4-b]pyridine-5-carboxylic acid ethyl ester,

1-(2-chloro-2-phenyl-ethyl)-4-cyclopropylamino-1H-pyrazolo[3,4-b]pyridine-5-carboxylic acid ethyl ester,

1-(2-chloro-2-phenyl-ethyl)-4-propylamino-1H-pyrazolo[3,4-b]pyridine-5-carboxylic acid ethyl ester,

1-(2-chloro-2-phenyl-ethyl)-4-phenylamino-1H-pyrazolo[3,4-b]pyridine-5-carboxylic acid ethyl ester,

4-butylamino-1-(2-chloro-2-phenyl-ethyl)-1H-pyrazolo[3,4-b]pyridine-5-carboxylic acid ethyl ester,

1-(2-chloro-2-phenyl-ethyl)-4-(2-ethoxy-ethylamino)-1H-pyrazolo[3,4-b]pyridine-5-carboxylic acid ethyl ester,

4-benzylamino-1-(2-chloro-2-phenyl-ethyl)-1H-pyrazolo[3,4-b]pyridine-5-carboxylic acid ethyl ester,

1-(2-chloro-2-phenyl-ethyl)-4-phenethylamino-1H-pyrazolo[3,4-b]pyridine-5-carboxylic acid ethyl ester,

Among the xanthine derivatives, one uses in particular (i) the (ω-1)-hydroxyalkyl-dialkylxanthines wherein the (ω-1)-hydroxyalkyl group contains 5 or 6 carbon atoms and is in position 1 or 7, the alkyl group in the other position 7 or 1 contains 1 to 12 carbon atoms and the alkyl group in position 3 contains 1 to 4 carbon atoms, (ii) the (ω-1)-oxoalkyl-dimethylxanthines wherein the (ω-1)-oxoalkyl group contains 5 or 6 carbon atoms and is in position 1 or 7, or (iii) derivatives of dimethylxanthine having an alkyl group containing from 4 to 12 carbon atoms or a benzyl group in position 1 or 7.

Typically, the oxoalkyl-dialkylxanthines include for example 1-(5-oxohexyl)-3,7- and 7-(5-oxohexyl)-1,3-dimethylxanthines. Other xanthines may also be used, such as in particular the 3,7-dimethylxanthines and 1,3-dimethylxanthines substituted with a butyl, isoamyl, hexyl, lauryl or benzyl group in position 1 or 7, as well as the homologues of these compounds with a hydroxy or oxo group in position (ω-1)-position, for example 1-(4-hydroxypentyl)- and 1-(5-hydroxyhexyl)-3,7-dimethylxanthines, 7-(4-hydroxypentyl)- and 7-(5-hydroxyhexyl)-1,3-dimethylxanthines, 1-(4-oxopentyl)-, 1-(5-oxohexyl)-, 1-(2-methyl-3-oxobutyl)- and 1-(2-ethyl-3-oxobutyl)-3,7-dimethylxanthines and the corresponding 1,3-dimethyl compounds having a (ω-1)-hydroxyalkyl or (ω-1)-oxoalkyl group in position 7. Homologues of the abovementioned hydroxyalkyl-dimethylxanthines are those having in position 1 or 7 which is not occupied by a hydroxyalkyl group, instead of a methyl group, an alkyl group having 2 to 12 carbon atoms, such as 1-ethyl-, 1-propyl-, 1-butyl- and 1-isobutyl-3-methyl-7-(5-hydroxyhexyl)-xanthines and 7-ethyl-, 7-propyl-, 7-butyl- and 7-isobutyl-1-(5-hydroxyhexyl)-3-methylxanthines, and the corresponding compounds having instead of a methyl group in position an alkyl group containing 2 to 4 carbon atoms, such as in particular an ethyl, n-propyl, isopropyl, isobutyl or n-butyl group.

Among such xanthine derivatives, one uses in particular pentoxifylline which has the following formula:

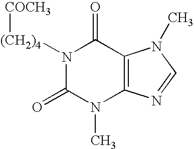

The present invention therefore proposes, for the first time, PDE4B as a therapeutic target for the treatment of molecular events associated with excitotoxicity. According to specific embodiments, the invention may be used to inhibit or reduce neuronal excitotoxicity in early stages of neurodegenerative diseases. It finds applications particularly in the treatment of Alzheimer's disease, Parkinson's disease, multiple sclerosis, ALS, Huntington's chorea and cerebral ischemia.

Other objects of the invention are based on:

use of the hereinabove compounds, particularly etazolate or pentoxifylline, for the treatment of ALS, notably to reduce neuronal excitotoxicity in the early stage of ALS, or

use of the hereinabove compounds, particularly pentoxifylline or etazolate, for preparing a composition designed to inhibit PDE4B activity in patients with ALS.

The invention equally concerns methods for treating ALS comprising administering a compound that selectively inhibits the expression or activity of PDE4B of sequence SEQ ID NO: 2 or 4. Preferably, the methods of the invention are used for treatment in the early stage of neurodegenerative diseases.

The administration may be performed by any method known to those skilled in the art, preferably by the oral route or by injection, typically by the intraperitoneal, intracerebral, intravenous, intraarterial or intramuscular route. Administration by the oral route is preferred. The administered doses may be adapted by those skilled in the art. Typically, approximately 0.01 mg to 100 mg/kg are injected, for inhibitor compounds that are chemical in nature. For nucleic compounds, doses may range for example from 0.01 mg to 100 mg per dose. It is understood that repeated injections may be given, possibly in combination with other active agents or any pharmaceutically acceptable vehicle (eg., buffers, isotonic saline solutions, in the presence of stabilisers, etc.).

The invention may be used in mammals, notably in human beings. The results presented in the examples illustrate the efficacy of PDE4B inhibitors in improving the viability of neurons placed in excitotoxicity conditions.

Methods of Selection and Tools

Other objects of the invention concern methods for selecting, identifying or characterising compounds active in diseases associated with excitotoxicity, or neuronal stress, comprising placing test compounds in contact with a cell expressing PDE4B (particularly a variant devoid of the 3′ noncoding region), and identifying compounds inhibiting the expression or activity of this protein.

The methods may be used with different cell populations, such as primary cells or cell lines of mammalian origin (human, murine, etc.). Advantageously, cells which do not naturally express PDE4B, transfected with a nucleic acid coding the desired variant, are used. In this manner, the selectivity of the method is increased. Lower eukaryotic cells (yeasts, etc.) or prokaryotic cells may also be used.

The screening methods may also be carried out in an acellular system, by measuring the capacity of test compounds to bind PDE4B or a variant or fragment thereof.

Another object of the invention concerns any nucleic acid coding a polypeptide such as defined hereinabove, vectors containing it, recombinant cells, and utilisations. The vectors may be plasmids, phages, cosmids, viruses, artificial chromosomes, etc. Preferred vectors are exemplified by plasmid vectors, such as those derived from commercially available plasmids (pUC, pcDNA, pBR, etc.). Such vectors advantageously contain a selection gene and/or an origin of replication and/or a transcriptional promoter. Other specific vectors are for example viruses or phages, particularly replication-defective recombinant viruses, such as viruses derived from retroviruses, adenoviruses, AAV, herpes virus, baculovirus, etc. The vectors may be used in any competent host, such as for example prokaryotic or eukaryotic cells. These may be bacteria (E. coli for example), yeasts (Saccharomyces or Kluyveromyces, for example), plant cells, insect cells, mammalian cells, notably human, etc. These may be cell lines, primary cells, mixed cultures, etc.

Other aspects and advantages of the present invention will become apparent from the following examples which are given for purposes of illustration and not by way of limitation.

### LEGENDS OF FIGURES

FIG. 1: Semi-quantitative PCR of PDE4B on brain (1A) and muscle (1B) specimens.

FIG. 2: Pentoxifylline protects primary neurons against formation of cerebellar granules related to excitotoxicity induced by kainate.

FIG. 3: Pentoxifylline protects primary neurons against formation of cerebellar granules related to excitotoxicity induced by NMDA/serine.

FIG. 4: Neuroprotective effect of etazolate against toxicity induced by NMDA/serine on brain granular cells.

FIG. 5: Neuroprotective effect of etazolate against toxicity induced by kainate on brain granular cells.

FIG. 6: Neuroprotective effect of pentoxifylline against toxicity induced by NMDA/serine on cortical neurons.

FIG. 7: Neuroprotective effect of pentoxifylline against toxicity induced by kainate on cortical neurons.

FIG. 8: Neuroprotective effect of etazolate against toxicity induced by NMDA/serine on cortical neurons.

FIG. 9: Neuroprotective effect of etazolate against toxicity induced by kainate on cortical neurons.

FIG. 10: Neuroprotective effect of 8-bromo-cAMP against toxicity induced by NMDA/serine on brain granular cells.

FIG. 11: Neuroprotective effect of 8-bromo-cAMP against toxicity induced by kainate on brain granular cells.

### EXAMPLES

### Example 1

### Identification of PDE4 as Molecular Target of Excitotoxicity

Qualitative differential analysis was carried out on polyadenylated (poly A+) RNA extracted from brain specimens of animals at different stages, without preliminary isolation of neurons so as to take into account a maximum of alternative splicing events linked to disease development.

Poly A+ RNAs are prepared by methods known to those skilled in the art. In particular, this may be a treatment by means of chaotropic agents such as guanidium thiocyanate followed by extraction of total RNA by means of solvents (phenol, chloroform for example). Such methods are well known to those skilled in the art [see Maniatis et al., Chomczynsli et al., Anal. Biochem. 162 (1987) 156], and may be easily practised by using commercially available kits. Poly A+ RNAs are prepared from these total RNAs according to conventional methods known to those skilled in the art and provided in commercially available kits.

These poly A+ RNAs serve as template for reverse transcription reactions using reverse transcriptase. In an advantageous manner, reverse transcriptases devoid of RNase H activity are used, so as to obtain first complementary DNA strands that are larger in size than those obtained with conventional reverse transcriptases. Such RNase H-free reverse transcriptase preparations are commercially available.

At each time point in disease development (30 days, 60 days and 90 days), the poly A+ RNAs as well as the single-stranded cDNAs are prepared from transgenic animals (T) and syngeneic control animals (C).

In accordance with the DATAS method, for each time point hybridisations are carried out of mRNA (C) with cDNA (T), and reciprocal hybridisations of mRNA (T) with cDNA (C).

The mRNA/cDNA heteroduplexes are then purified according to the protocols of the DATAS method.

RNA sequences not paired with a complementary DNA are released from these heteroduplexes through the action of RNAse H, as this enzyme degrades paired RNA sequences. Such unpaired sequences represent qualitative differences existing between RNAs which by the way are homologous between themselves. These qualitative differences may be located anywhere on the RNA sequence, at the 5′ or 3′ region or inside the sequence and notably in the coding sequence. Depending on their location, these sequences may not only be alternative splicings, but also may be the result of translocations or deletions.

The RNA sequences representing qualitative differences are then cloned according to methods known to those skilled in the art and more specifically those described in the patent for the DATAS method.

Such sequences are entered into cDNA banks which constitute qualitative differential banks. One such bank contains the exons and introns specific of the healthy situation; the other banks contain the splicing events characteristic of pathological conditions.

Differential expression of the clones was checked by hybridisation with probes obtained by reverse transcription of messenger RNAs extracted from the different situations under study. Clones showing differential hybridisation were retained for subsequent analysis. The sequences identified by DATAS correspond to introns and/or exons differentially expressed through splicing in pathological situations and in the healthy situation. These splicing events may be specific of a given stage in the development of the disease or characteristic of the healthy state.

Comparison of these sequences with databases makes it possible to classify the information obtained and propose a reasoned selection of sequences according to their diagnostic or therapeutic interest.

The performance of DATAS on RNAs from 60-day-old transgenic and control animals has led to the isolation of a cDNA fragment derived from phosphodiesterase 4B mRNA. This fragment corresponds to an exon fragment specifically present in control animals and therefore specifically deleted in SOD1G93A transgenic animals at the 60-day stage. The fragment covers nucleotides 377 to 486 numbered from the stop codon of murine PDE4B (SEQ ID NO:1). This sequence comprises 2912 bases, the deleted fragment corresponding to bases 2760 to 2869. This region is noncoding and is expressed differentially between control animals and transgenic animals, due to alternative use of a 3′ noncoding exon or due to the use of two alternative polyadenylation sites.

### Example 2

### RT-PCR Experiments: Confirmation of Differential Expression

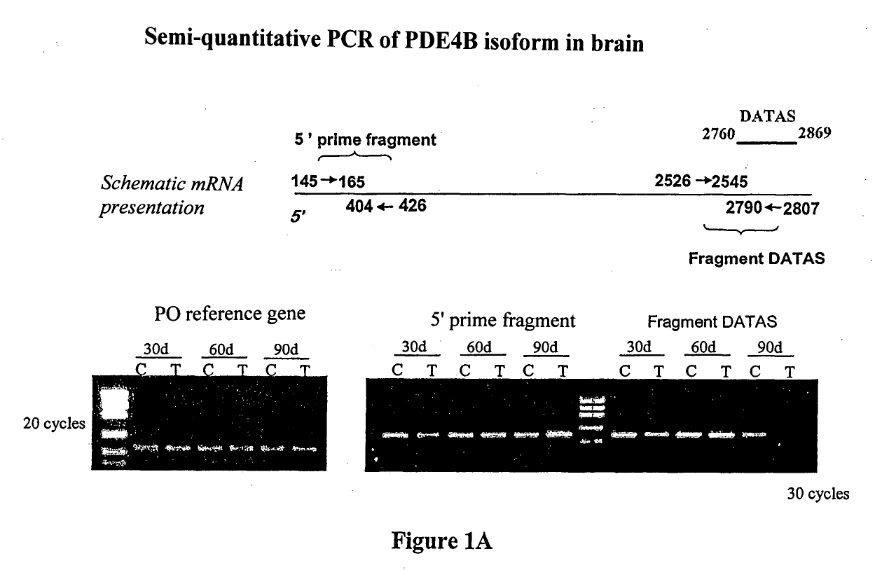

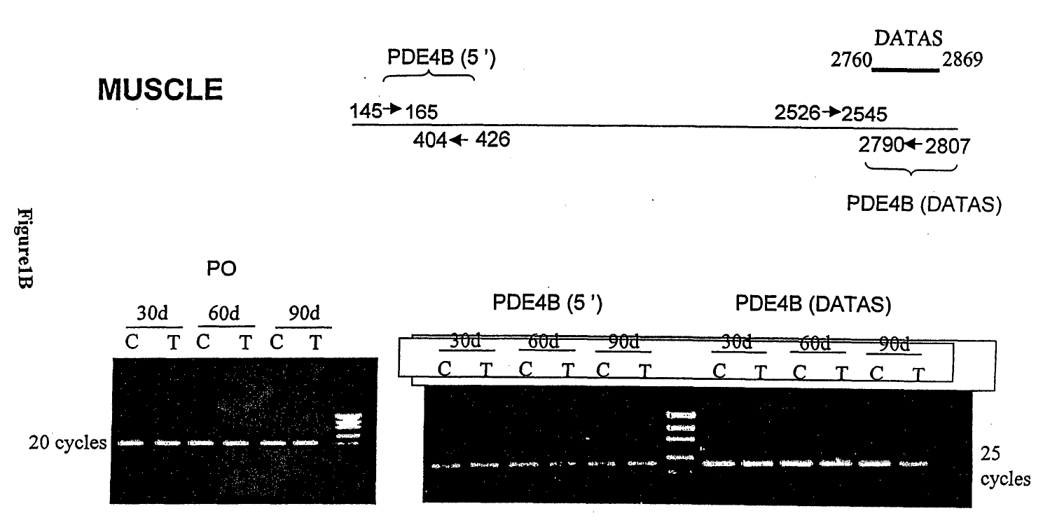

Differential expression of PDE4B in a situation of neuronal stress, as compared to a reference situation, was verified by the RT-PCR experiments described in FIG. 1.

These experiments were conducted according to methods well known to those skilled in the art and made it possible to follow the expressions of two distinct regions of PDE4B mRNA. One such region spans the initiation codon of this mRNA (PDE4B 5′), the other partly spans the fragment identified by the DATAS method (PDE4B DATAS). The locations of the PCR primers used are indicated in FIG. 1.

PO RNA is a ribosomal RNA serving as internal control to check that the same amount of RNA was used for each experimental point. Analyses were performed with RNA extracted from control (C) and transgenic (T) animals aged 30, 60 and 90 days, i.e. before onset of pathological symptoms.

Total RNAs from the brains of control or SOD1 G93A mice aged 30, 60 or 90 days were transcribed to cDNA using the standard Superscript™ protocol (Invitrogen). For semi-quantitative PCR the reverse transcription reaction products were diluted ten-fold. The specific primers of the DATAS fragment correspond to nucleotides 2526 to 2545 for the sense strand (5′ GCC AGG CCG TGA AGC AAA TA 3′; SEQ ID NO: 5), and to nucleotides 2790 to 2807 for the antisense strand (5′ TCA MG ACG CGA AAA CAT 3′; SEQ ID NO: 6) and for the more 3 prime fragment the primers correspond to nucleotides 145 to 165 for the sense strand (5′ CCG CGT CAG TGC CTT TGC TAT 3′; SEQ ID NO: 7), and to nucleotides 426 to 404 for the antisense strand (5′ CGC TGT CGG ATG CTT TTA TTC AC 3′; SEQ ID NO: 8). The P0 gene was used as reference and amplified by the following primers: sense strand: 5′ TCG CTT TCT GGA GGG TGT C 3′ (SEQ ID NO: 9) and antisense strand: CCG CAG GGG CAG CAG TGG 3′ (SEQ ID NO:10).

Amplification was achieved by 30 PCR cycles as follows:

30 seconds at 94° C.

1 minute at 57° C.

30 seconds at 72° C., followed by a cycle of 2 minutes at 72° C.

The different PCR products were loaded on a 1.5% agarose gel. The experience was carried out in triplicate with two different reverse transcription reactions.

FIG. 1 shows the results obtained from RNAs extracted from brain or muscle of the animals.

Whereas the same quantity of cDNA is amplified from PO RNA in all samples, variations are seen with PDE4B mRNA. The most significant variations are detected in the 90-day-old animals: while an increase in the expression of the PDE4 5′ fragment is observed in brain of transgenic animals, a very strong decrease in PDE4B (DATAS) expression occurs in the brain of transgenic animals.

This finding establishes a correlation between the decrease in expression of a 3′ noncoding mRNA fragment of PDE4B and the increase in expression of the 5′ coding region of this same messenger. This result is altogether compatible with the presence of mRNA destabilising sequences in the sequence identified by DATAS and demonstrates the correlation between PDE4B expression and the phenomenon of excitotoxicity.

### Example 3

### Inhibition of Excitotoxicity by Inhibitors of PDE4

For this example, rat brain granular as well as cortical neurons were cultured according to techniques known to those skilled in the art.

Primary Rat Brain Granular Cell Cultures:

Seven-day-old Wistar rats were decapitated and their brains dissected. After removing the meninges, the tissue was cut into small pieces and trypsinized for 15 minutes at 37° C. The cells were dissociated in a grinder and seeded at a density of 300,000 cells per cm² into basic Eagle medium supplemented with 10% fetal calf serum and 2 mM glutamine. The next day, 10 μM ARA-C, an antimitotic agent, was added to inhibit the growth of glial cells. After nine days of culture, cells were treated with the phosphodiesterase inhibitors pentoxifylline and etazolate, three hours before adding the toxins 50 μM kainate or 100 μm N-methyl-D-aspartate in the presence of 10 μM D-serine. 8-bromo-cAMP was added immediately before the toxins. All treatments were performed at least in duplicate and in at least two different cultures. After a 6 hour incubation, toxicity was evaluated by an MTT test. The results, normalized to the mean of untreated controls, were analysed statistically with a Wilcoxon test. The level of significance was set at p<0.05.

Primary Cortical Cell Cultures:

Sixteen-day-old embryos from Wistar rats were removed and the cortex dissected. After trypsinization for 25 minutes at 37° C., the cells were dissociated in a grinder, then seeded at a density of 300,000 cells per cm² into minimum essential medium supplemented with 10% horse serum, 10% fetal calf serum and 2 mM glutamine. After four days of culture, half of the medium was replaced by minimum essential medium supplemented with 5% horse serum and 2 mM glutamine. On the same day, 10 μM 5-fluoro-2-deoxyuridine, an antimitotic agent, was added. After 7 and 11 days of culture, half of the medium was replaced by conditioned medium composed of MEM supplemented with 5% horse serum and 2 mM glutamine; this medium was passed overnight on a layer of cortical astrocytes before use. On day 14, cells were treated with the phosphodiesterase inhibitors pentoxifylline and etazolate 1 hour before adding the toxins 50 μM kainate or 20 μM N-methyl-D-aspartate in the presence of 10 μM D-serine. All treatments were performed at least in duplicate and in at least two different cultures. After a 6 hour incubation, toxicity was evaluated by an MTT test. The results, normalized to the mean of untreated controls, were analysed statistically with a Wilcoxon test. The level of significance was set at p<0.05.

MTT:

Toxicity was measured with an MTT test. After incubation with the compounds, MTT was added at 0.5 mg/ml final concentration per well. Plates were then incubated for 30 minutes at 37° C. in the dark. The medium was aspirated and the crystals resuspended in 500 μl of DMSO (dimethylsulfoxide). Absorbance at 550 nm was read and the percentage viability was calculated.

Results:

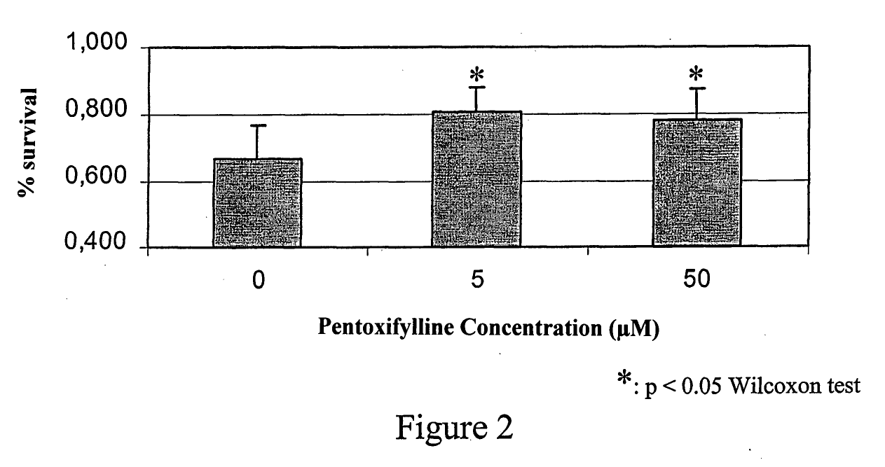

The results are presented in FIGS. 2 to 10. These results illustrate the protective effect of the compounds according to the invention on neuron survival. When neurons were cotreated with a PDE4 inhibitor, a dose-dependent protective effect was observed with both excitotoxicity inducers (NMDA/serine and kainate). Such a protective effect was observed with pentoxifylline and etazolate.

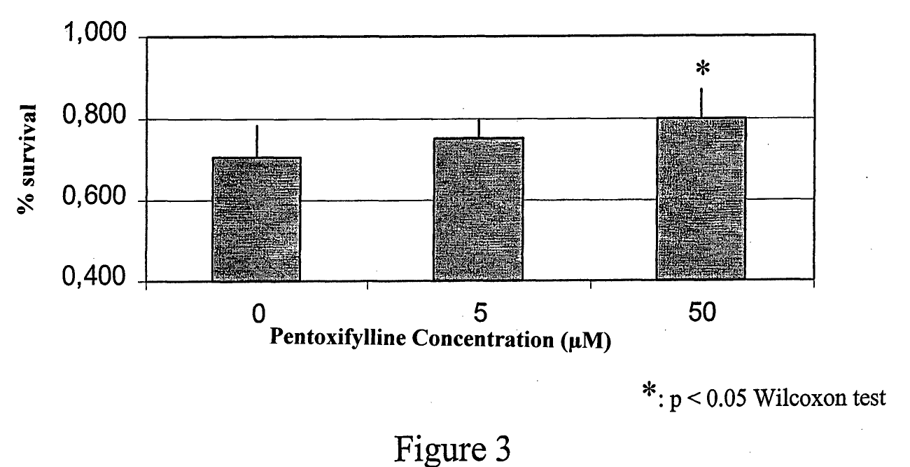

FIGS. 2 and 3 show the results obtained with pentoxifylline on brain granular cells. They show that pentoxifylline affords 43% protection of these cells in the case of NMDA/serine treatment, and 33% in the case of kainate-induced toxicity.

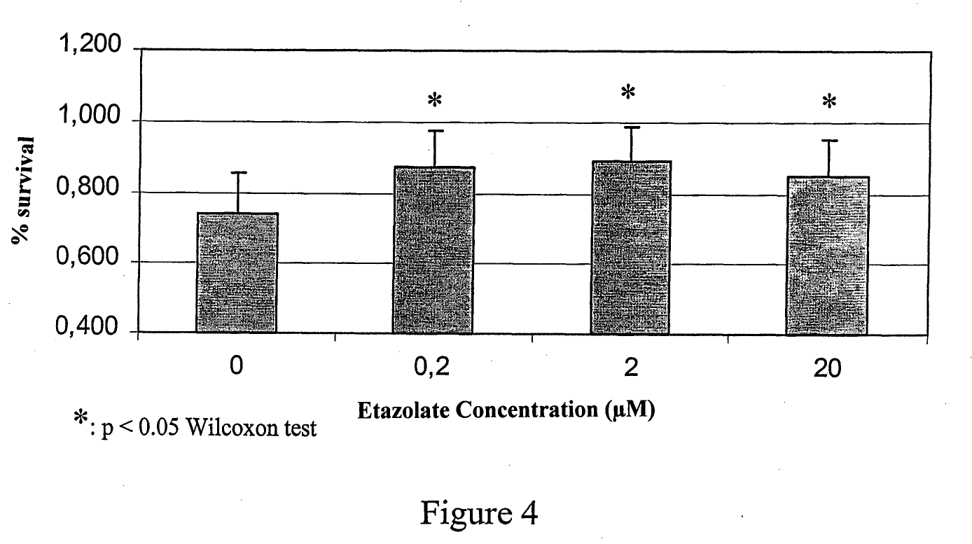

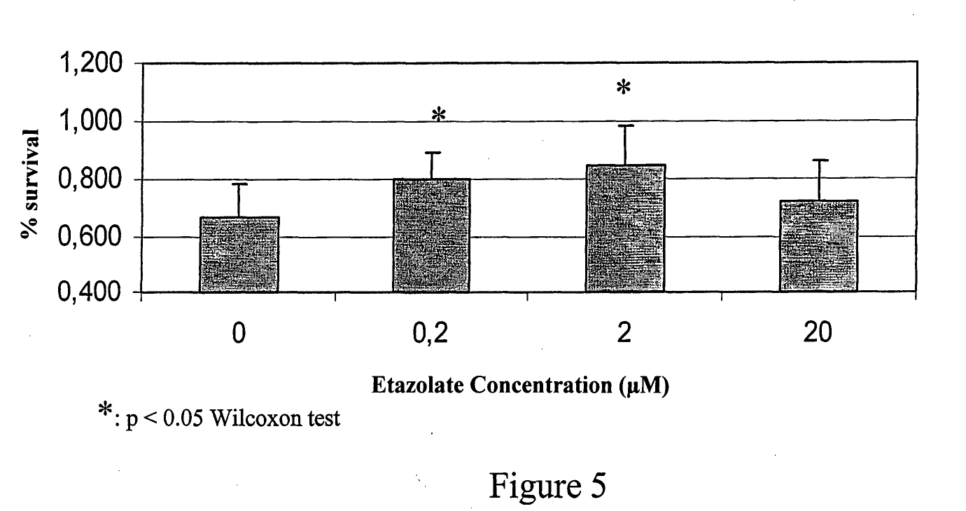

FIGS. 4 and 5 present the results obtained with etazolate on brain granular cells. They show that etazolate gives 60% protection of these cells in the case of NMDA/serine treatment, and 57% in the case of kainate-induced toxicity.

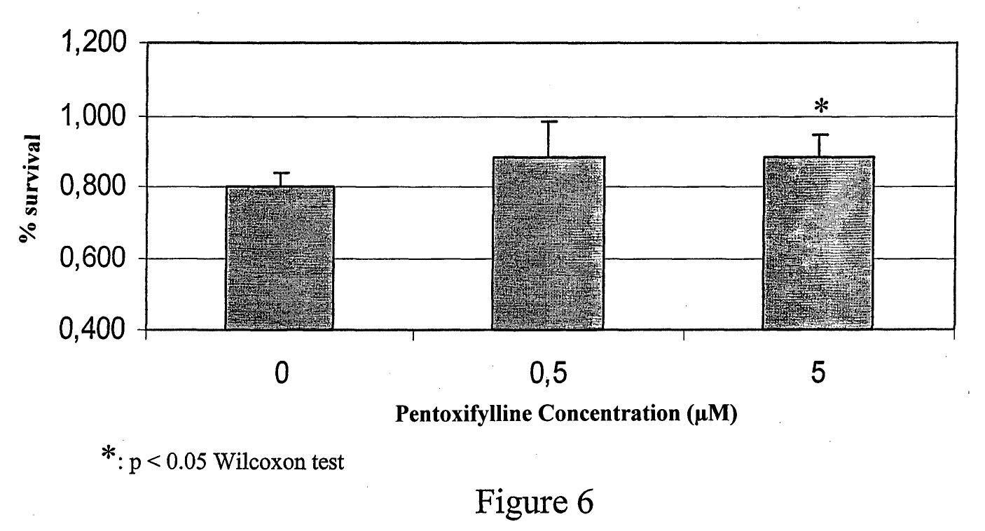

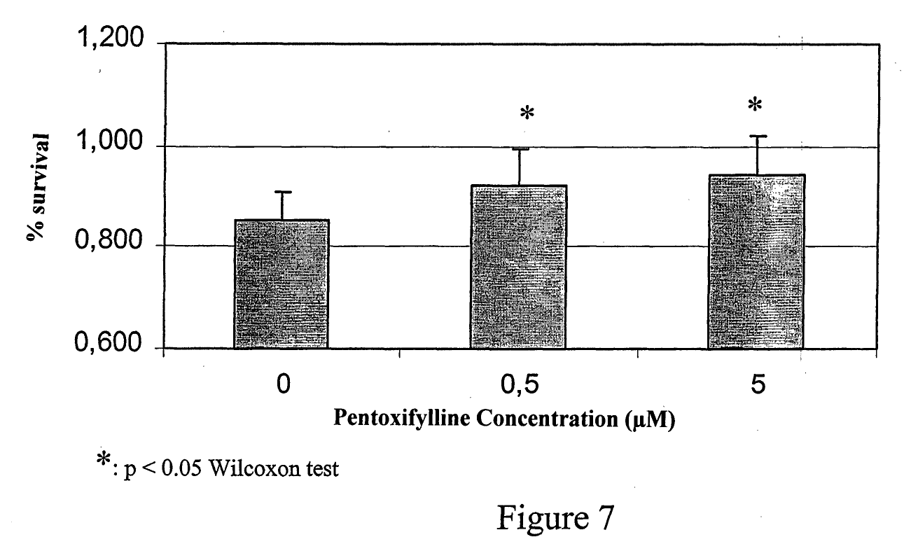

FIGS. 6 and 7 show the results obtained with pentoxifylline on cortical neurons. They show that pentoxifylline affords a 50% protective effect on these cells in the case of NMDA/serine treatment, and 66% in the case of kainate-induced toxicity.

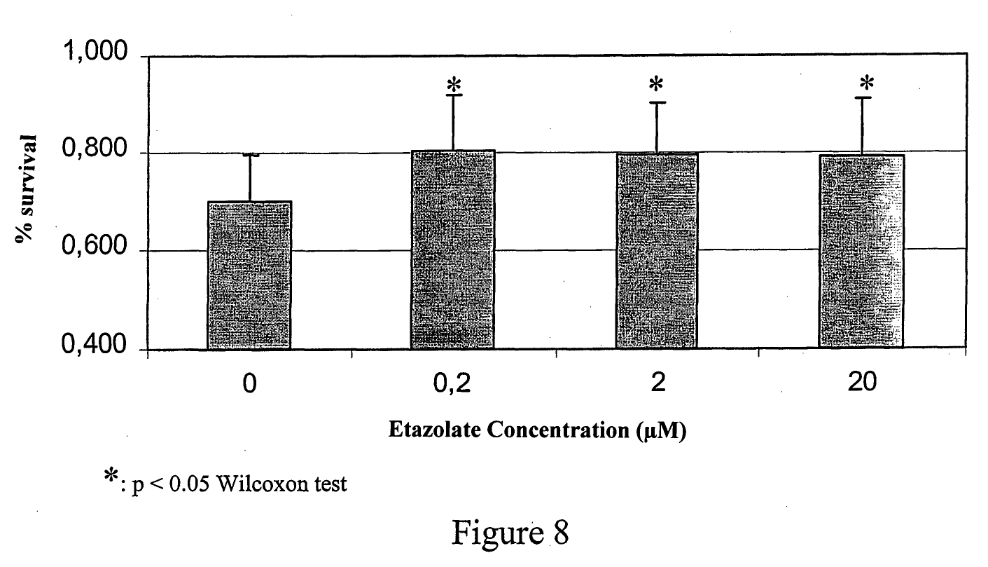

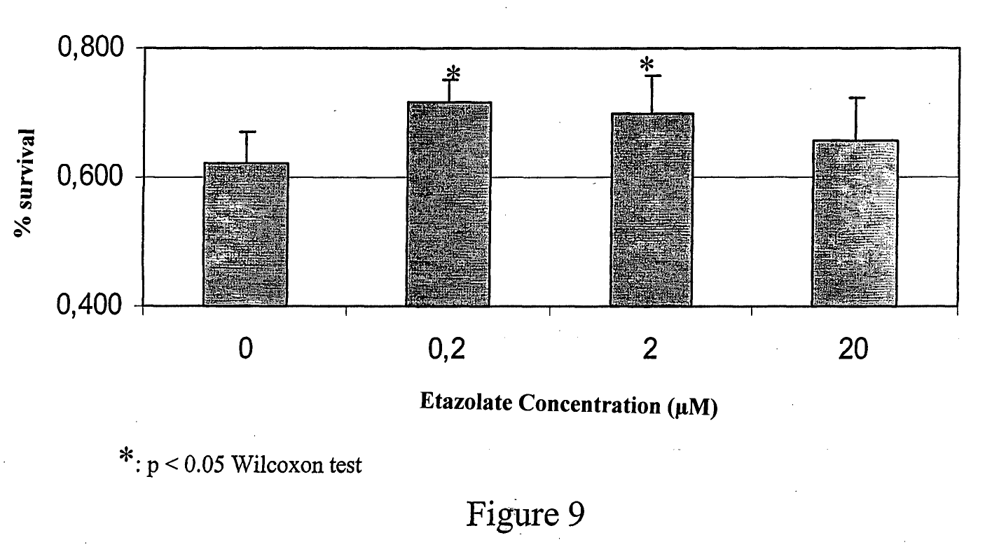

FIGS. 8 and 9 give the results obtained with etozalate on cortical neurons. They show that etozalate provides 33% protection of these cells in the case of NMDA/serine treatment, and 25% in the case of kainate-induced toxicity.

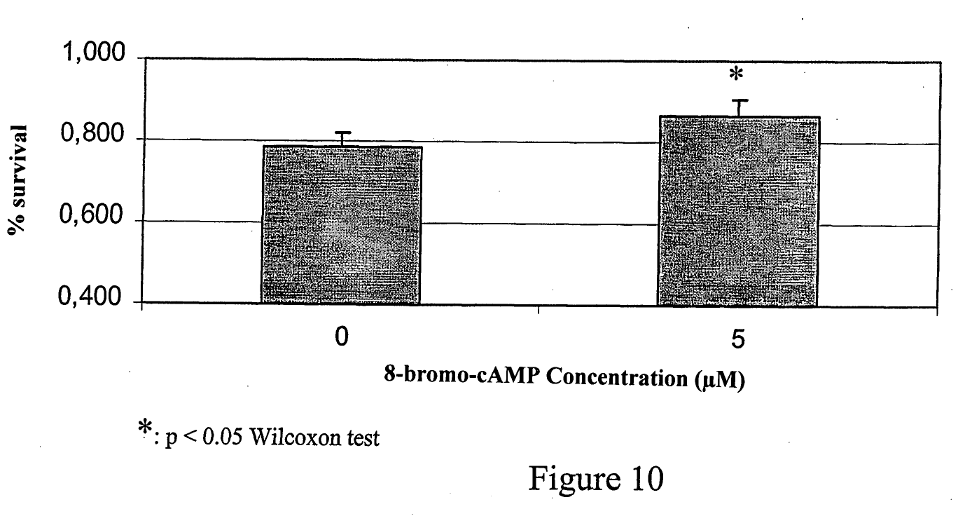

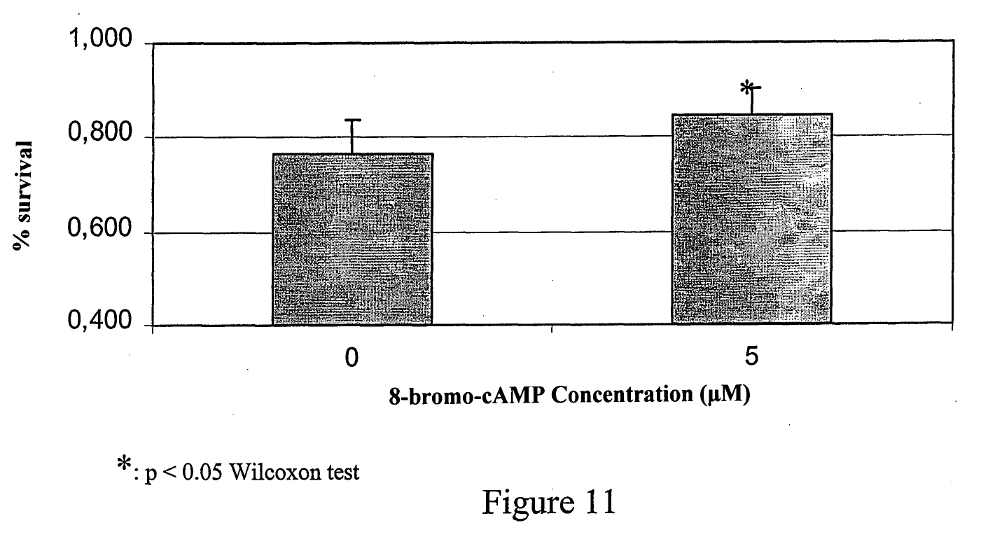

The relevance of this protection is confirmed by the percent of protection achieved with increasing concentrations of cAMP, a PDE substrate, given as an example for brain granular cells in FIGS. 10 and 11. A 40% protection was observed for NMDA/serine treatment and 40% with kainate treatment.

The present invention therefore not only demonstrates the involvement of PDE4B in mechanisms of excitotoxicity, particularly in an ALS model, but also demonstrates the ability of PDE4 inhibitors to preserve neuronal viability during stress linked to excitotoxicity.

### Example 4

### Clinical use in Man

This example describes the conditions of human clinical use of a PDE4 inhibitor in the treatment of ALS. This example illustrates the therapeutic potential of the invention and its conditions of implementation in man.

In this clinical trial, treatment is based on a combination of pentoxifylline and riluzole, the former at a dose of 400 mg three times a day, for a total daily dose of 1200 mg. Pentoxifylline is administered as a tablet formulation. This is a multicenter, double-blind, placebo-controlled trial in 400 patients comprising men and women aged 18 to 80 years, presenting with sporadic or familial ALS, and on therapy with riluzole (50 mg b.i.d.) for at least 3 months. The projected duration of treatment with pentoxifylline is 18 months.

The main efficacy endpoints are survival rate, quality of life and muscle tests.

Other aspects and applications of the invention concern:

use of all or part of a sequence derived from PDE4B messenger RNA for purposes of diagnosis or screening or characterisation of neurodegenerative diseases having a component or a stage related to the excitotoxicity phenomenon, such as Alzheimer's disease, Parkinson's disease, multiple sclerosis, Huntington's chorea, ALS or cerebral ischemia,

use of any nucleic acid fragment including antisense RNAs for purposes of inhibiting expression of PDE4B in patients with such diseases,

use of any chemical compound, particularly pentoxifylline, etazolate, or any pharmaceutical composition containing them, for purposes of inhibiting PDE4B activity in patients with such diseases,

use of all or part of a sequence derived from PDE4B messenger RNA for purposes of characterising tissue and the ischemic situation.

1-19. canceled.

20. A method for detecting a situation of excitotoxicity or neuronal stress in a subject, comprising measuring in vitro the expression of phosphodiesterase 4, particularly phosphodiesterase 4B, in a sample from the subject.

21. A method according to claim 20, comprising detecting the presence of a mutant RNA of phosphodiesterase 4, particularly phosphodiesterase 4B, in a sample from the subject, in particular a form deleted of all or part of the 3′ noncoding region.

22. A method according to claim 20, comprising the placing in contact in vitro of a biological sample from a subject, containing a nucleic acid, with a nucleic acid comprising all or part of a sequence derived from the PDE4B messenger RNA or gene, and detecting the formation of a hybrid.

23. A method according to claim 20, comprising the placing in contact in vitro of a biological sample from a subject, containing a nucleic acid, with a nucleic acid comprising all or part of a sequence derived from the PDE4B messenger RNA or gene and wherein the sample comprises nerve or muscle cells.

24. A method according to claim 20, for the diagnosis or detection of Alzheimer's disease, Parkinson's disease, multiple sclerosis, Huntington's chorea, ALS or cerebral ischemia.

25. A method for the treatment of diseases associated with neuronal stress, particularly excitotoxicity, comprising administering to a subject in need of such treatment a compound that inhibits PDE4B activity or expression, preferably a compound that selectively inhibits PDE4.

26. A method according to claim 25, wherein the neuronal excitotoxicity is associated with a neurodegenerative disease.

27. A method according to claim 25, wherein the compound is an antisense nucleic acid, capable of inhibiting transcription of the PDE4B gene or translation of the corresponding messenger.

28. A method according to claim 25, wherein the compound is a chemical compound of natural or synthetic origin.

29. A method according to claim 25, wherein the compound is chosen from among the compounds in the pyrazolopyridine family, particularly etazolate, and the compounds in the family of xanthine derivatives, particularly pentoxifylline.

30. A method according to claim 25, to inhibit or reduce neuronal excitotoxicity in the early stage of neurodegenerative diseases.

31. A method according to claim 25, to improve neuron survival in patients with ALS.

32. A method according to claim 25, for the treatment of Alzheimer's disease, Parkinson's disease, multiple sclerosis, Huntington's chorea or cerebral ischemia.

33. A method according to claim 25, for the treatment of ALS, particularly to reduce neuronal excitotoxicity in the early stage of ALS.

34. A method according to claim 25, wherein the compound is pentoxifylline.

35. A method according to claim 34, to increase neuron survival in patients with ALS.

36. A method according to claim 25, wherein the compound is etazolate.

37. A method according to claim 36, to increase neuron survival in patients with ALS.

38. A method according to claim 25, wherein the compound is selected from the group consisting of:
4-butylamino-1-ethyl-6-methyl-1H-pyrazolo[3,4-b]pyridine-5-carboxylic acid ethyl ester (tracazolate),
4-butylamino-1-ethyl-1H-pyrazolo[3,4-b]pyridine-5-carboxylic acid ethyl ester,
1-(4-amino-pyrazolo[3,4-b]pyridin-1-yl)-□-D-1-deoxy-ribofuranose,
1-ethyl-4-(N′-isopropylidene-hydrazino)-1H-pyrazolo[3,4-b]pyridine-5-carboxylic acid ethyl ester (SQ 20009),
4-amino-6-methyl-1-n-pentyl-1H-pyrazolo[3,4-b]pyridine,
4-amino-1-ethyl-6-methyl-1H-pyrazolo[3,4-b]pyridine-5-carboxylic acid ethyl ester (desbutyl tracacolate),
4-amino-1-pentyl-1H-pyrazolo[3,4-b]pyridine-5-carboxamide,
1-ethyl-6-methyl-4-methylamino-1H-pyrazolo[3,4-b]pyridine-5-carboxylic acid ethyl ester,
4-amino-6-methyl-1-propyl-1H-pyrazolo[3,4-b]pyridine-5-carboxylic acid ethyl ester,
1-ethyl-4-ethylamino-6-methyl-1H-pyrazolo[3,4-b]pyridine-5-carboxylic acid ethyl ester,
4-amino-1-butyl-6-methyl-1H-pyrazolo[3,4-b]pyridine-5-carboxylic acid ethyl ester,
5-(4-amino-pyrazolo[3,4-b]pyridin-1-yl)-2-hydroxymethyl-tetrahydro-furan-3-ol,
1-allyl-4-amino-6-methyl-1H-pyrazolo[3,4-b]pyridine-5-carboxylic acid allyl ester,
4-amino-6-methyl-1-pentyl-1H-pyrazolo[3,4-b]pyridine-5-carboxylic acid,
4-amino-1-ethyl-3,6-dimethyl-1H-pyrazolo[3,4-b]pyridine-5-carboxylic acid ethyl ester,
4-dimethylamino-1-ethyl-6-methyl-1H-pyrazolo[3,4-b]pyridine-5-carboxylic acid ethyl ester,
1-ethyl-6-methyl-4-propylamino-1H-pyrazolo[3,4-b]pyridine-5-carboxylic acid ethyl ester,
4-amino-1-pentyl-1H-pyrazolo[3,4-b]pyridine-5-carboxylic acid ethyl ester,
4-amino-6-methyl-1-pent-4-ynyl-1H-pyrazolo[3,4-b]pyridine-5-carboxylic acid ethyl ester,
4-amino-1-but-3-enyl-1H-pyrazolo[3,4-b]pyridine-5-allylamide,
4-amino-1-pentyl-1H-pyrazolo[3,4-b]pyridine-5-isopropylamide,
4-amino-1-pentyl-N-n-propyl-1H-pyrazolo-[3,4-b]pyridine-5-carboxamide,
4-amino-1-butyl-6-methyl-1H-pyrazolo[3,4-b]pyridine-5-carboxylic acid allyl ester,
4-amino-6-methyl-1-pent-3-ynyl-1H-pyrazolo[3,4-b]pyridine-5-carboxylic acid ethyl ester,
4-amino-1-pentyl-1H-pyrazolo[3,4-b]pyridine-5-prop-2-ynylamide,
4-amino-1-(3-methyl-butyl)-1H-pyrazolo[3,4-b]pyridine-5-carboxylic acid allyl ester,
4-amino-1-pentyl-1H-pyrazolo<3,4-b>pyridine-5-N-(2-propenyl)carboxamide,
4-amino-1-pentyl-1H-pyrazolo[3,4-b]pyridine-5-carboxylic acid allyl ester,
4-amino-1-pentyl-1H-pyrazolo[3,4-b]pyridine-5-butylamide,
4-amino-1-but-3-ynyl-6-methyl-1H-pyrazolo[3,4-b]pyridine-5-carboxylic acid allyl ester,
4-amino-1-but-3-enyl-6-methyl-1H-pyrazolo[3,4-b]pyridine-5-carboxylic acid allyl ester,
4-amino-6-methyl-1-pentyl-1H-pyrazolo[3,4-b]pyridine-5-allylamide,
4-amino-6-methyl-1-pentyl-1H-pyrazolo[3,4-b]pyridine-5-carboxylic acid allyl ester,
4-amino-6-methyl-1-(3-methyl-butyl)-1H-pyrazolo[3,4-b]pyridine-5-carboxylic acid allyl ester,
4-amino-6-methyl-1-pentyl-1H-pyrazolo[3,4-b]pyridine-5-carboxylic acid isobutyl ester,
4-amino-6-methyl-1-pentyl-1H-pyrazolo[3,4-b]pyridine-5-butylamide,
4-amino-6-methyl-1-(3-methyl-but-2-enyl)-1H-pyrazolo[3,4-b]pyridine-5-carboxylic acid allyl ester,
4-amino-1-pentyl-1H-pyrazolo[3,4-b]pyridine-5-cyclopropylamide,
ethyl 4-amino-1-pentyl-1H-pyrazolo[3,4-b]pyridine-5-hydroxamate,
4-amino-6-methyl-1-pentyl-1H-pyrazolo[3,4-b]pyridine-5-carboxylic acid prop-2-ynyl ester,
4-amino-6-methyl-1-pent-4-ynyl-1H-pyrazolo[3,4-b]pyridine-5-carboxylic acid allyl ester,
4-amino-6-methyl-1-pent-4-enyl-1H-pyrazolo[3,4-b]pyridine-5-carboxylic acid allyl ester,
4-amino-1-pent-3-ynyl-1H-pyrazolo[3,4-b]pyridine-5-propylamide,
4-amino-1-pentyl-1H-pyrazolo[3,4-b]pyridine-5-cyclopropylmethyl-amide,
4-amino-6-methyl-1-pentyl-1H-pyrazolo[3,4-b]pyridine-5-carboxylic acid 2-methyl-allyl ester,
4-amino-1-pent-3-ynyl-1H-pyrazolo[3,4-b]pyridine-5-allylamide (ICI 190,622),
4-amino-1-pent-4-ynyl-N-2-propenyl-1H-pyrazolo[3,4-b]pyridine-5-carboxamide,
4-amino-1-pent-3-ynyl-1H-pyrazolo[3,4-b]pyridine-5-prop-2-ynylamide,
4-amino-1-pentyl-1H-pyrazolo[3,4-b]pyridine-5-but-2-ynylamide,
4-amino-6-methyl-1-pent-3-ynyl-1H-pyrazolo[3,4-b]pyridine-5-carboxylic acid allyl ester,
4-amino-1-(2-cyclopropyl-ethyl)-6-methyl-1H-pyrazolo[3,4-b]pyridine-5-carboxylic acid allyl ester,
4-amino-1-hex-5-ynyl-6-methyl-1H-pyrazolo[3,4-b]pyridine-5-carboxylic acid allyl ester,
4-amino-1-pent-3-ynyl-1H-pyrazolo[3,4-b]pyridine-5-cyclopropylmethyl-amide,
4-amino-6-methyl-1-pentyl-1H-pyrazolo[3,4-b]pyridine-5-carboxylic acid but-3-enyl ester,
4-amino-6-methyl-1-pentyl-1H-pyrazolo[3,4-b]pyridine-5-carboxylic acid cyclopropylmethyl ester,
4-butylamino-1-pentyl-1H-pyrazolo[3,4-b]pyridine-5-allylamide,
4-amino-6-methyl-1-pentyl-1H-pyrazolo[3,4-b]pyridine-5-carboxylic acid 2-cyclopropyl-ethyl ester,
4-amino-6-methyl-1-pent-3-ynyl-1H-pyrazolo[3,4-b]pyridine-5-carboxylic acid cyclopropylmethyl ester,
4-amino-6-methyl-1-pent-4-ynyl-1H-pyrazolo[3,4-b]pyridine-5-carboxylic acid cyclopropylmethyl ester,
4-amino-1-benzyl-6-methyl-1H-pyrazolo[3,4-b]pyridine-5-carboxylic acid ethyl ester,
4-amino-1-pentyl-1H-pyrazolo[3,4-b]pyridine-5-benzylamide,
4-amino-1-pentyl-1H-pyrazolo[3,4-b]pyridine-5-phenylamide,
4-amino-6-methyl-1-pentyl-1H-pyrazolo[3,4-b]pyridine-5-carboxylic acid benzyl ester,
4-azido-1-□-D-ribofuranosylpyrazolo[3,4-b]pyridine,
1-pent-3-ynyl-N-2-propenyl-4-propionamido-1 H-pyrazolo[3,4-b]pyridine-5-carboxamide,
2-(4-amino-pyrazolo[3,4-b]pyridin-1-yl)-5-hydroxymethyl-tetrahydro-furan-3,4-diol,
2-(6-methyl-1H-pyrazolo[3,4-b]pyridin-4-ylamino)-ethanol,
3-(6-methyl-1H-pyrazolo[3,4-b]pyridin-4-ylamino)-propan-1-ol,
3-(6-methyl-1H-pyrazolo[3,4-b]pyridin-4-ylamino)-acetic acid propyl ester,
2-(6-methyl-1H-pyrazolo[3,4-b]pyridin-4-ylamino)-propionic acid ethyl ester,
2-(6-methyl-1H-pyrazolo[3,4-b]pyridin-4-ylamino)-pentanoic acid ethyl ester,
2-(6-methyl-1H-pyrazolo[3,4-b]pyridin-4-ylamino)-benzoic acid ethyl ester,
3-(6-methyl-1H-pyrazolo[3,4-b]pyridin-4-ylamino)-pentanoic acid propyl ester,
N-benzylidene-N′-(3-methyl-1-phenyl-1H-pyrazolo[3,4-b]pyridin-4-yl)-hydrazine,
N-furan-2-ylmethylene-N′-(3-methyl-1-phenyl-1H-pyrazolo[3,4-b]pyridin-4-yl)-hydrazine,
N-(4-fluoro-benzylidene)-N′-(3-methyl-1-phenyl-1H-pyrazolo[3,4-b]pyridin-4-yl)-hydrazine,
N-(3-furan-2-yl-allylidene)-N′-(3-methyl-1-phenyl-1H-pyrazolo[3,4-b]pyridin-4-yl)-hydrazine,
N-(4-methoxy-benzylidene)-N′-(3-methyl-1-phenyl-1H-pyrazolo[3,4-b]pyridin-4-yl)-hydrazine,
4-[(3-methyl-1-phenyl-1H-pyrazolo[3,4-b]pyridin-4-yl)-hydrazonomethyl]-benzonitrile,
N-benzo[1,3]dioxol-5-ylmethylene-N′-(3-methyl-1-phenyl-1H-pyrazolo[3,4-b]pyridin-4-yl)-hydrazine,
N-(3-methyl-1-phenyl-1H-pyrazolo[3,4-b]pyridin-4-yl)-N′-(4-nitro-benzylidene)-hydrazine,
N-(3-methyl-1-phenyl-1H-pyrazolo[3,4-b]pyridin-4-yl)-N′-(2-nitro-benzylidene)-hydrazine,
N-(3-methyl-1-phenyl-1H-pyrazolo[3,4-b]pyridin-4-yl)-N′-(4-trifluoromethyl-benzylidene)-hydrazine,
N-(3-methyl-1-phenyl-1H-pyrazolo[3,4-b]pyridin-4-yl)-N′-(5-nitro-furan-2-ylmethylene)-hydrazine,
N-(3-methyl-1-phenyl-1H-pyrazolo[3,4-b]pyridin-4-yl)-N′-(2-trifluoromethyl-benzylidene)-hydrazine,
N-(3-methyl-1-phenyl-1H-pyrazolo[3,4-b]pyridin-4-yl)-N′-(6-nitro-benzo[1,3]dioxol-5-ylmethylene)-hydrazine,
4-(3-chloro-4-methoxy-benzylamino)-1-ethyl-1H-pyrazolo[3,4-b]pyridine-5-carboxylic acid,
4-(3-chloro-4-methoxy-benzylamino)-1-ethyl-1H-pyrazolo[3,4-b]pyridine-5-(pyridin-4-ylmethyl)-amide,
4-(3-chloro-4-methoxy-benzylamino)-1-ethyl-1H-pyrazolo[3,4-b]pyridine-5-(tetrahydro-furan-2-ylmethyl)-amide,
4-(3-chloro-4-methoxy-benzylamino)-1-ethyl-1H-pyrazolo[3,4-b]pyridine-5-(5-hydroxy-pentyl)-amide,
4-(3-chloro-4-methoxy-benzylamino)-1-ethyl-1H-pyrazolo[3,4-b]pyridine-5-[3-(2-oxo-pyrrolidin-1-yl)-propyl]-amide,
4-tert-butylamino-1-(2-chloro-2-phenyl-ethyl)-1H-pyrazolo[3,4-b]pyridine-5-carboxylic acid ethyl ester,
1-(2-chloro-2-phenyl-ethyl)-4-cyclopropylamino-1H-pyrazolo[3,4-b]pyridine-5-carboxylic acid ethyl ester,
1-(2-chloro-2-phenyl-ethyl)-4-propylamino-1H-pyrazolo[3,4-b]pyridine-5-carboxylic acid ethyl ester,
1-(2-chloro-2-phenyl-ethyl)-4-phenylamino-1H-pyrazolo[3,4-b]pyridine-5-carboxylic acid ethyl ester,
4-butylamino-1-(2-chloro-2-phenyl-ethyl)-1H-pyrazolo[3,4-b]pyridine-5-carboxylic acid ethyl ester,
1-(2-chloro-2-phenyl-ethyl)-4-(2-ethoxy-ethylamino)-1H-pyrazolo[3,4-b]pyridine-5-carboxylic acid ethyl ester,
4-benzylamino-1-(2-chloro-2-phenyl-ethyl)-1H-pyrazolo[3,4-b]pyridine-5-carboxylic acid ethyl ester, and
1-(2-chloro-2-phenyl-ethyl)-4-phenethylamino-1H-pyrazolo[3,4-b]pyridine-5-carboxylic acid ethyl ester.

39. A method according to claim 25, wherein the compound is a PDE4 inhibitor compound belonging to the pyrazolopyridine family, to increase neuron survival in patients with ALS.

40. Nucleotide primer, wherein it is composed of a fragment of 8 to 20 consecutive residues from the sequence located between nucleotides 2384 and 2869 of sequence SEQ ID NO: 1 or between nucleotides 2461 and 4068 of the sequence SEQ ID NO: 3 or a sequence complementary thereto.

41. Kit for analysing PDE4 expression, particularly differential expression between the 3′ noncoding region and the coding region, the kit comprising a nucleotide probe specific of part of the sequence of the 3′ noncoding region and a nucleotide probe specific of part of the sequence of the coding region.

42. Kit for analysing PDE4 expression, particularly differential expression between the 3′ noncoding region and the coding region, the kit comprising a pair of nucleotide primers allowing specific amplification of at least part of the 3′ noncoding region of PDE4 and a pair of nucleotide primers allowing specific amplification of at least part of the PDE4 coding region.


SEQUENCE LISTING (10 sequences)

SEQ ID NO: 1 - DNA, 2912 bp, Mus musculus
  CDS (218)..(2383)
aaaggcagcctgataaagctccttgtgacaggctgtcttgccagtctcccagtatgctcctcttgctctgaagtgctccaggattgaaaccacagcttcccaaattagcctgggaagagtgtgcggacccagcagccttttaacccgcgtcagtgcctttgctatgttcaagactgctgttttggatggtgaatgctagctagcactccatcgagacatgacagcaaaaaattctccaaaagaatttactgcttcggaatctgaggtttgcataaagactttcaaggagcagatgcgcttggaacttgagcttccaaagctaccaggaaacagacctacatctcccaaaatttctccacgcagttcaccaaggaattcaccatgctttttcagaaagttgctggtgaataaaagcatccgacagcggcgtcgcttcacggtggctcatacatgctttgatgtggaaaatggcccttctccaggtcggagcccactggaccctcaagccggctcttcgtcgggactggtacttcatgccgcctttcctgggcacagccagcgcagggagtcgttcctctacgatcttgacagcgactatgacttgtcaccaaaagcgatgtccaggaactcatcacttcccagtgagcaacacggcgatgacctgattgtcactccttttgcccaggttcttgccagcttgcgaagtgtaagaaacaacttcaccctgctgacgaaccttcatggagcgccgaacaagaggtcaccagcggctagtcaggctccagtctccagagtcagcctgcaagaggaatcatatcagaaactagcaatggagacgctggaggaactagactggtgcctagaccagctagagaccatccagacctaccgctctgtcagcgagatggcttcaaacaagttcaaaaggatgctgaaccgggagctgacacacctctcagagatgagcagatcagggaaccaggtgtctgagtacatttcaaacacgttcttagacaagcagaacgatgtggaaatcccatctcccacgcagaaggacagggagaagaagaagaagcagcagctcatgacccagataagtggagtgaagaaactgatgcacagctcaagcctgaacaacacaagcatctcacgcttcgggatcaacacggaaaatgaggatcatctagccaaggagctggaagacctgaacaaatggggccttaacatcttcaatgtggctgggtactcacataatcggccccttacgtgcatcatgtatgcaatattccaggaaagagaccttctgaagacgtttaaaatctcatctgacacctttgtaacctacatgatgactttagaagaccattaccattctgatgtggcatatcacaacagcctgcatgctgctgacgtggcccagtcaactcacgttctcctttctacgccggcactggatgctgtcttcacagacctggaaatcctggctgccatttttgcagctgccatccatgatgtcgatcatcctggagtctccaatcagtttctcatcaatacaaattctgaacttgctttgatgtataatgatgaatctgttctggaaaaccatcaccttgctgtgggattcaaattgctacaagaggaacactgcgacatctttcagaatcttaccaagaagcaacgccagacactcaggaaaatggtgattgacatggtgttggcaactgatatgtccaaacacatgagcctcctggcagaccttaaaacaatggtagaaaccaagaaggtgacaagctccggtgttctcctcctggacaactatactgaccggatacaggttcttcgcaacatggtacactgtgcagacctgagcaaccccaccaagtccttggaattgtatcggcaatggaccgatcgtatcatggaggagtttttccagcagggagacaaagaacgggagaggggaatggagattagcccaatgtgtgataagcacacagcttctgtggaaaaatcccaggttggtttcattgactacattgtccatccactgtgggagacctgggcagacctggttcaaccggatgctcaagatattctggatacactagaagataacaggaactggtaccagagtatgataccccagagcccttccccgccactggatgagaggagcagggactgccaaggcctgatggagaagtttcagtttgaactgacccttgaggaagaggattctgagggaccggaaaaggagggagaaggccacagctatttcagcagcacaaagacgctttgtgtgattgatccagagaacagggattctctggaagagactgacatagacattgcaacagaagacaagtctccgatcgacacataatctctctccctctgtgtggagatgaacattccacccttgactgagcatgcccgctgagtggtagggtcacctaccatggccaaggcctgcacaggacaaaggccacctggcctttccagttacttgagtttggagccagaatgccaggccgtgaagcaaatagcagttccatgctgtcttgccttgcctgcaagcttggcggagacccgcagctgtatgtggtagtagaggccagttcccatcaaagctaaaatggcttgaaaacagaggacacaaagctgagagattgctctgcactaggtgttgggaagctgtcctgacagatgactgaactcactaacaacttcatctataaatctcaccacccaacccattgtctgccaacctgtgtgcctttttttgtaaaatgttttcgcgtctttgaaatgcctgttgaatatctagagtttagtaccaacttctacaaacttttttgagtctttcttgaaaaacaaaaaaaaaaaaaaaaaaaaaaaaaaaaaaaaaaaaa

SEQ ID NO: 2 - protein, 721 aa, Mus musculus
MTAKNSPKEFTASESEVCIKTFKEQMRLELELPKLPGNRPTSPKISPRSSPRNSPCFFRKLLVNKSIRQRRRFTVAHTCFDVENGPSPGRSPLDPQAGSSSGLVLHAAFPGHSQRRESFLYDLDSDYDLSPKAMSRNSSLPSEQHGDDLIVTPFAQVLASLRSVRNNFTLLTNLHGAPNKRSPAASQAPVSRVSLQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGINTENEDHLAKELEDLNKWGLNIFNVAGYSHNRPLTCIMYAIFQERDLLKTFKISSDTFVTYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFQNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDERSRDCQGLMEKFQFELTLEEEDSEGPEKEGEGHSYFSSTKTLCVIDPENRDSLEETDIDIATEDKSPIDT

SEQ ID NO: 3 - DNA, 4068 bp, Homo sapiens
  CDS (766)..(2460): PDE4B
gaattcctcctctcttcaccccgttagctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttcacccgaattttgactgtttcatttagaagaaaagcaaaatgagaaaaagctttcctcatttctccttgagatggcaaagcactcagaaatgacatcacataccctaaagaaccctgggatgactaaggcagagagagtctgagaaaactctttggtgcttctgcctttagttttaggacacatttatgcagatgagcttataagagaccgttccctccgccttcttcctcagaggaagtttcttggtagatcaccgacacctcatccaggcggggggttggggggaaacttggcaccagccatcccaggcagagcaccactgtgatttgttctcctggtggagagagctggaaggaaggagccagcgtgcaaataatgaaggagcacgggggcaccttcagtagcaccggaatcagcggtggtagcggtgactctgctatggacagcctgcagccgctccagcctaactacatgcctgtgtgtttgtttgcagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgccccccacaggagttgctacagtccctggccctgcttcccatcctcctctcttcaccccgttagctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttggaaacaatattctcacattagatactaaatggtttatactgagtcttttacttttgtatagcttgataggggcaggggcaatgggatgtagtttttacccaggttctatccaaatctatgtgggcatgagttgggttataactggatcctactatcattgtggctttggttcaaaaggaaacactacatttgctcacagatgattcttctgattcttctgaatgctcccgaactactgactttgaagaggtagcctcctgcctgccattaagcaggaatgtcatgttccagttcattacaaaagaaaacaataaaacaatgtgaatttttataataaaaaaaaaaaaaaggaattc

SEQ ID NO: 4 - protein, 564 aa, Homo sapiens
MKEHGGTFSSTGISGGSGDSAMDSLQPLQPNYMPVCLFAEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDEQNRDCQGLMEKFQFELTLDEEDSEGPEKEGEGHSYFSSTKTLCVIDPENRDSLGETDIDIATEDKSPVDT

SEQ ID NO: 5 - DNA, 20 bp, Artificial Sequence
  Description of Artificial Sequence primer
gccaggccgtgaagcaaata

SEQ ID NO: 6 - DNA, 18 bp, artificial sequence
  Description of Artificial Sequence primer
tcaaagacgcgaaaacat

SEQ ID NO: 7 - DNA, 21 bp, artificial sequence
  Description of Artificial Sequence primer
ccgcgtcagtgcctttgctat

SEQ ID NO: 8 - DNA, 23 bp, artificial sequence
  Description of Artificial Sequence primer
cgctgtcggatgcttttattcac

SEQ ID NO: 9 - DNA, 19 bp, artificial sequence
  Description of Artificial Sequence primer
tcgctttctggagggtgtc

SEQ ID NO: 10 - DNA, 18 bp, artificial sequence
  Description of Artificial Sequence primer
ccgcaggggcagcagtgg